(19) United States
(12) Patent Application Publication
(10) Pub. No.: US 2002/0107556 A1
(43) Pub. Date: Aug. 8, 2002

(54) Movement initiation device used in Parkinson's disease and other disorders which affect muscle control

(76) Inventors: Raphael Fifo Mcloul, Atlanta, GA (US); Caryn Oberman, Atlanta, GA (US)

Correspondence Address: BERNSTEIN & ASSOCIATES, PC, 6600 PEACHTREE DUNWOODY RD, NE, EMBASSY ROW 400, SUITE 495, ATLANTA, GA, 30328-1649, US

(21) Appl. No.: 10/022,961

(22) Filed: Dec. 13, 2001

Related U.S. Application Data
(60) Provisional application No. 60/255,617, filed on Dec. 13, 2000.

(51) Int. Cl. A61N 1/10

(52) U.S. Cl. 607/48

(57) ABSTRACT

A portable wearable device that produces rhythmic stimuli in the form of auditory, visual, tactile, and or vibratory activity that initiates or assists in the continuing movement of the body's muscles that have a tendency to become rigid and immobile in patients with certain diseases, such as Parkinson's Disease and Parkinsonism. Components include a housing, a clinician-accessible controller, a user-accessible controller, a transducer for producing sound or vibratory signals, and, a computer interface.

### CROSS-REFERENCE TO RELATED APPLICATION

This application claims benefit of copending U.S. provisional application No. 60/255,617, filed Dec. 13, 2001, entitled “Movement Initiation Device Used In Parkinson's Disease and Other Disorders Which Affect Muscle Control,” the disclosure of which is incorporated in its entirety herein by reference. This application also relates to Disclosure Document No. 452,955, filed Mar. 15, 1999, entitled “Device for Treatment of Movement-Initiation Problems in Parkinson's Disease Patients,” which is incorporated in its entirety herein by reference.

### FIELD OF THE INVENTION

The present invention relates to a portable, hand-holdable or body wearable, programmable device that produces rhythmic stimuli in the form of auditory, visual, tactile, and or vibratory activity that initiates or assists in the continuing movement of the body's muscles that have a tendency to become rigid and immobile in patients with certain diseases, such as Parkinson's Disease and Parkinsonism.

### BACKGROUND OF THE INVENTION

Even the smallest muscular movement requires information and complex decisions involving the many different areas of the brain. This information comes together in a central part of the brain, known as the striatum, which initiates and controls human bodily motion. The striatum works with other areas of the brain, substantially with an area called the substantial nigra, to send commands for balance, coordination, and movement These commands are sent from the brain to the spinal cord through a network of nerve cells to the muscles within the body. The central nervous system is made up entirely of individual nerve cells that actually serve as a communication network within the body. Nerve cells communicate with one another through the use of chemical messengers called neurotransmitters, which carry messages between nerve cells by leaping across the space, or synapse, between the cells. These links between the brain and the muscles allow thought to be translated into motion.

An especially important neurotransmitter is dopamine, which is manufactured by the substantial nigra for transmitting messages to the striatum. Another important messenger, acetylcholine, works in conjunction with dopamine to produce balance and smooth, controlled movement. Some of the nerve cells in the brain are specialized to use either dopamine or acetylcholine to send different messages, depending on what it is that an individual wants to do. The nerve cells that produce dopamine release dopamine particles through an opening into the synapse adjacent to a dopamine receiving nerve cell. The dopamine particles flow across the synapse and settle within a pocket along the outside of a receiving nerve cell, which is then stimulated to pass along the message to the next receiving nerve cell. Once a receiving cell has been stimulated to pass on the message, the used dopamine, as well as any excess dopamine, is then released back into the synapse, where it is broken down by a chemical in the synapse. This step also assists in the precise control of muscle movement, because too much or too little dopamine can interfere with smooth and continuous motion. The nerve cells that produce acetylcholine release acetylcholine particles and stimulate the acetylcholine receiving nerve cells in much the same way as do the dopamine particles with respect to the dopamine receiving nerve cells. When one decides to make a specific movement, the brain understands what kind of movement is desired and sends out a balance of dopamine and acetylcholine to enable one to move in a smooth and controlled manner. With the presence of Parkinson's disease, dopamine producing nerve cells begin to diminish, causing a decrease in the amount of available dopamine. The acetylcholine producing nerve cells, however, are not affected by the disease and the imbalance of dopamine and acetylcholine within the brain results in a lack of coordination of the body's movements.

Parkinson's disease is a chronic nervous disorder that affects an estimated eight million people worldwide. In many patients the symptoms of Parkinson's disease have an identifiable cause, in which cases the syndrome is known as Parkinsonism. Such symptoms include tremors, slowness of motion, muscle immobility, a lack of muscle control, and a state of imbalance. As described above, these symptoms occur when living cells are destroyed in certain parts of the brain and when there is a deficiency of dopamine, an amino acid produced by living cells within the body. After a number of years of enduring such symptoms, one may develop a stooped posture and slow gait which can be erratic and unsteady. Additionally, the muscles will become rigid, preventing even the simplest of movements. Often the rigidity of facial muscles causes speech impairment.

While there is still no cure for Parkinson's disease and no way to restore the lost nerve cells that produce dopamine or reverse its course in any way, several treatment options exist that can be used to control or alleviate the symptoms associated with the disease. Medications that can increase the level of dopamine in the brain and correct the imbalance of dopamine with respect to acetylcholine are currently available. Many of the pharmaceutical treatments developed over the years have been somewhat successful in treating the symptoms associated with Parkinson's disease and Parkisonism, but for many patients, not without a decrease in its effectiveness over time and not without occurrence of side effects that cause neurologic and psychiatric disturbances. For example, the brain may receive too much dopamine from medication, causing dyskinesia, a distortion or impairment of voluntary movement (such as a tic or spasm). Additionally, pharmaceutical treatment has often been ineffective in relieving gait defects, despite the overall effectiveness of the dopamine therapy. Another treatment option is surgical treatment that transplants dopamine producing tissue or growth factor producing cells into the brain to regrow damaged dopamine producing nerve cells. Unfortunately, the positive effects of such surgical treatment are limited, lasting only up to five years.

It is generally thought that patients who have undergone surgery or are being treated through drug therapy need effective adjunctive therapies to maximize the relief of symptoms and to further improve mobility. Thus, alternative therapies, including exercise and diet, physical therapy, music therapy, and external rhythmic stimulation from aural, visual, vibratory, and or tactile cues, have been designed to elicit motor responses in neurologically impaired patients without significant side effects and have been tested to determine their effectiveness. Of these, external rhythmic stimulation, has proven the most effective, enabling patients to walk faster, take longer strides and better improve their overall motor timing than with physical therapy or other non-rhythmic stimulation. Although rhythmic sensory cueing in general can be used to stabilize motor control and attain performance improvements, rhythmic auditory cues consistently create substantially faster physical response times than do visual or tactile cues. It is also thought that listening to music woven with rhythmic auditory tones may be even more beneficial for the patients.

With the application of music and or rhythmic auditory therapy, it has been found that the motor system of the human body is physiologically sensitive to arousal by the auditory system. Rhythmic auditory patterns stimulate neural motor impulses based on the attraction of the neurotransmitters to the auditory signal frequency. This profound effect of rhythmic patterns on the motor system strongly suggests that the timing of music and other auditory sounds is the most essential element relating rhythmic auditory stimulation to motor behavior. Thus, rhythmic stimuli in general, whether it is auditory, vibratory, visual or tactile, seem to act as external clocks to which the body synchronizes its motion and steps to take over the time keeping and rhythm formation processes that are unstable patients with Parkinson's disease and other disorders accompanied by impaired motor function. As a result, patients are able to synchronize their movement patterns to the rhythmic cues emanating from a source such as a metronome. Patients undergoing rhythmic auditory therapy may be able to decrease the dosage of their medication and delay surgery for up to five years. Even patients that are not taking medication to increase the amount of dopamine levels within their bodies have been able to benefit significantly from rhythmic auditory response training. Patients having been exposed to rhythmic auditory stimulation, either independently or as an adjunct therapy, have more stable swallowing patterns, increased speed and stability in motor functions, and an easier time with daily walking activities.

Presently, rhythmic stimulation is administered by a physician or physical therapist in an office or hospital setting with the proper equipment. For example, rhythmic auditory stimulation is administered with the use of a metronome. Gains from rhythmic stimuli are generally maintained for approximately one month. Therefore, the patient must make monthly visits to receive this treatment. This routine can be very inconvenient and costly. Thus, there is a great need for a portable and either hand-holdable or body wearable device that can be easily programmed by the physician, the patient or by both to produce the rhythms, whether they be auditory, vibratory, visual, tactile or a combination thereof, that will elicit the desired motor response for a particular individual.

### SUMMARY OF THE INVENTION

The present invention provides an apparatus and method for dealing with neurological disorders. A feature of the present invention is to assist the human body with the initiation and maintenance of continuous movement. A second feature of the present invention is to provide rhythmic stimuli to enhance a person's ability to control their motor functions. Another feature calls for the rhythmic stimuli to be audio signals within a prescribed range of parameters. A further feature is for the present invention to be programmable for independent users and customized for each patient's needs.

Generally described, the present invention comprises a wearable, portable, and/or hand-holdable, programmable device and system used in the treatment of Parkinson's disease and other disorders which affect muscle control. The device produces rhythmic stimuli in the form of auditory, visual, tactile, and/or vibratory activity that initiates or assists in continuing movement of the body's muscles that have a tendency to become rigid and immovable in patients with diseases, symptoms, and/or syndromes such as Parkinson's Disease and Parkinsonism.

The apparatus of the present invention disclosed herein comprises two or more main components that work together to generate a desired output: an output device (auditory or other), and either or both a user controller and a clinician controller. The output device transmits or projects the actual rhythmic stimuli to the patient. The user controller is a device used by the patient, caregiver, or physician that exerts limited control over the output of the rhythmic stimuli based on programmed parameters. The clinician controller uniquely programs the user controller and/or the output unit to produce the desired stimuli. Each component may be separately housed or may be integrated into a single or dual housing.

The primary modality for treatment of symptoms is for the device of the present invention to use rhythmic auditory stimulation to improve impairments of movement including but not limited to decreased gait velocity, incorrect or inconsistent cadence, shortened stride length, insufficient heelstrike and toe clearance, inadequate flexion, other impaired locomotor activity, and impaired control of deliberate limb movements resulting from postural instability, disequilibrium, or muscle rigidity. In this case, the output device is envisioned in the form of a hearing aid or similar device consisting of an input, a central processing unit, memory, clock, synthesizer, and a transducer that outputs the rhythmic auditory stimuli. The user controller is envisioned as a hand-held, tabletop, or wearable device consisting of a user interface, central processing unit, memory, wired or wireless input, and output that sends signals, commands, and/or programs to the output device. The clinician controller is envisioned as a hand-held or tabletop unit, or as a personal computer or other computing device that includes appropriate user interface, a central processing unit, memory and an output that sends signals, commands and/or programs to the user controller and/or the output device.

Alternative embodiments of this invention include using rhythmic visual, tactile, and/or vibratory stimuli to treat similar symptoms as described previously. Other features and advantages of the present invention will become apparent upon reading the following detailed description of embodiments of the invention, when taken in conjunction with the appended claims.

### BRIEF DESCRIPTION OF THE DRAWINGS

The invention is illustrated in the drawings in which like reference characters designate the same or similar parts throughout the figures of which:

FIG. 1 is a schematic diagram of the system overview showing relationship between clinician controller, user controller, audio output unit and the auditory system of the patient being treated.

FIG. 2 is a flow diagram of clinician controller.

FIG. 3 is a schematic diagram of user controller.

FIG. 4 is a schematic diagram of audio output unit.

FIG. 5 is a clinician controller, user controller, and audio output integrated into a single hand held or table top unit.

FIG. 6 is a tabletop or hand holdable clinician controller.

FIG. 7 is a hand held user controller.

FIG. 8 is a user controller integrated into a single wearable or pocket-fitting unit.

FIG. 9 is a watch style user controller.

FIG. 10 is a clip on (pager style) user controller.

FIG. 11 is an earpiece audio output unit positioned about the ear.

FIG. 12 is a front view of earpiece audio output unit.

FIG. 13 is a back view of earpiece audio output unit.

FIG. 14 is a small clip-on audio output unit.

FIG. 15 is a user controller and audio output integrated into a single hand held unit.

FIG. 16 is an audio output unit integrated into a single earpiece with limited controls, such as on/off and volume controls.

FIG. 17 is a user controller and audio output unit integrated into a single wearable or pocket-fitting unit.

FIG. 18 is an audio and visual cue combination unit comprising a pair of eyeglasses with speaker and visual display.

### DETAILED DESCRIPTION OF THE INVENTION

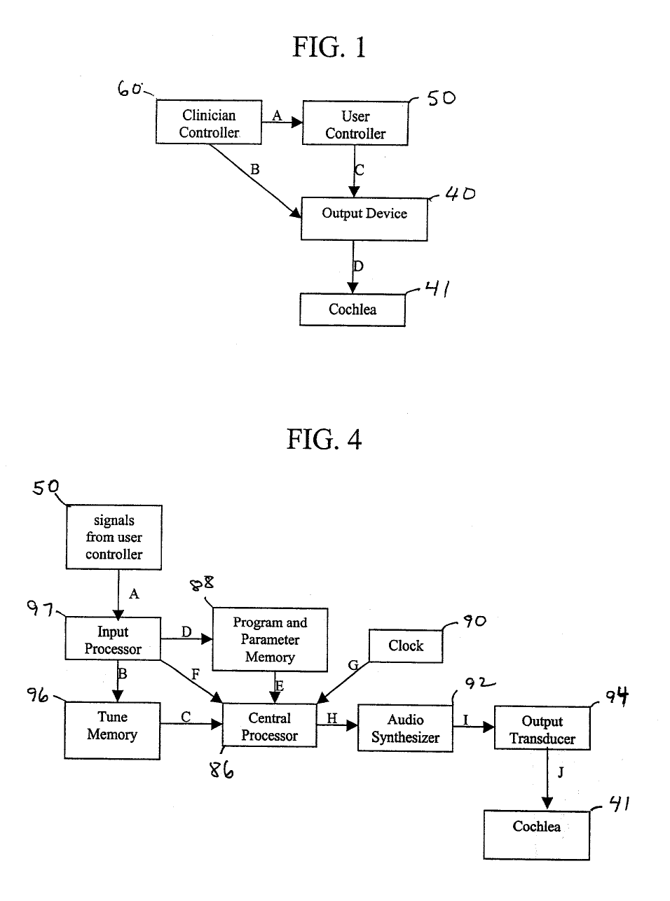

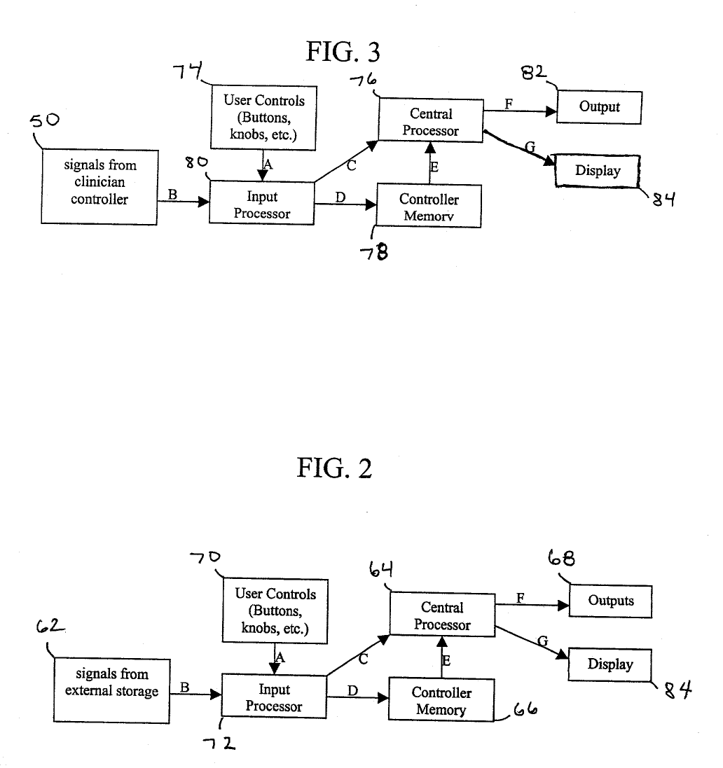

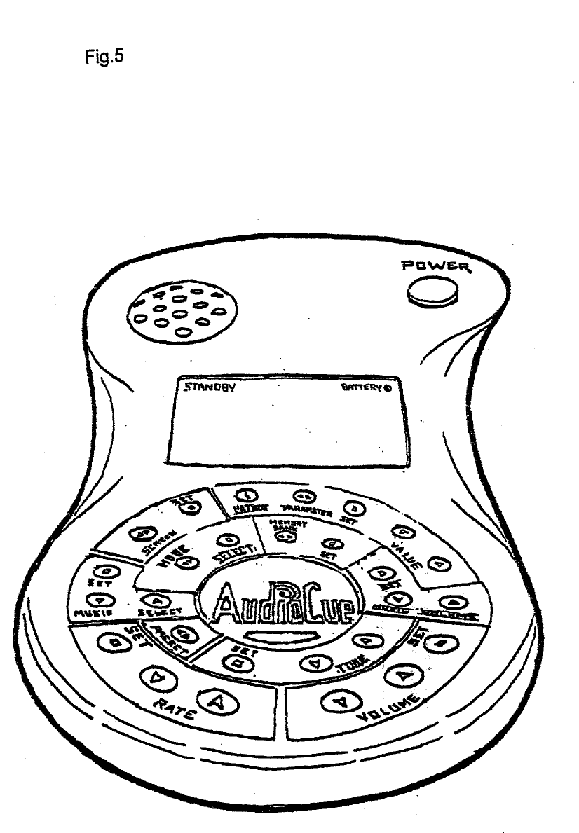

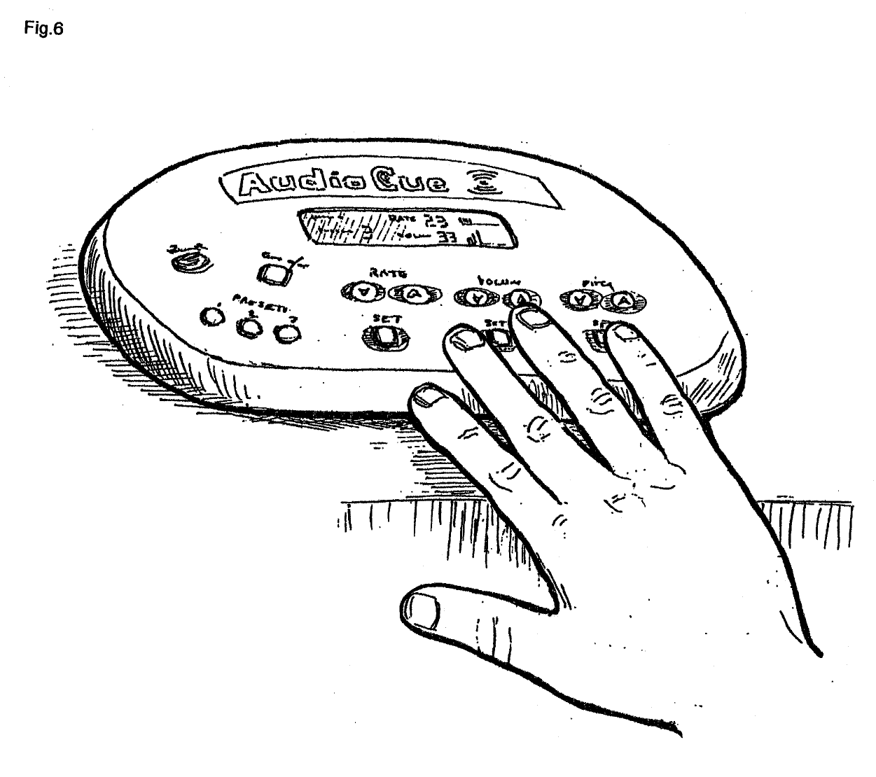

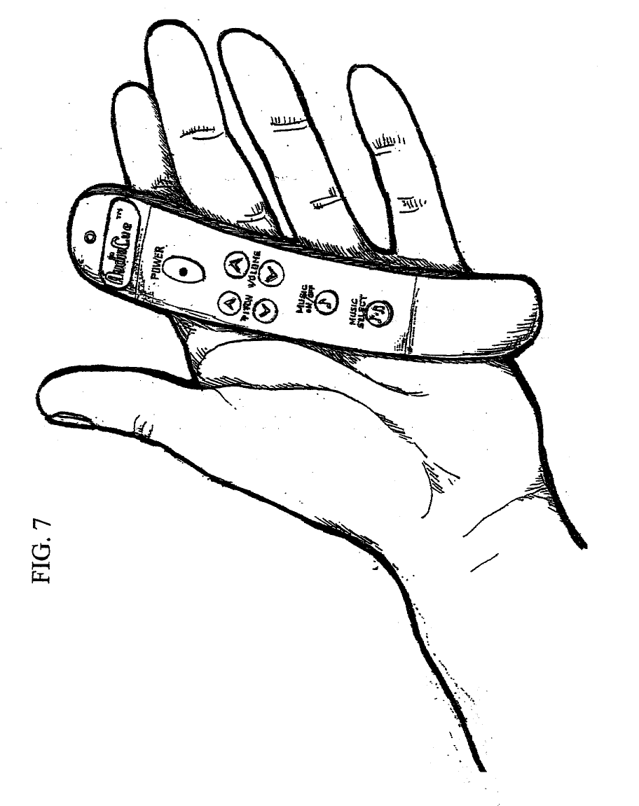

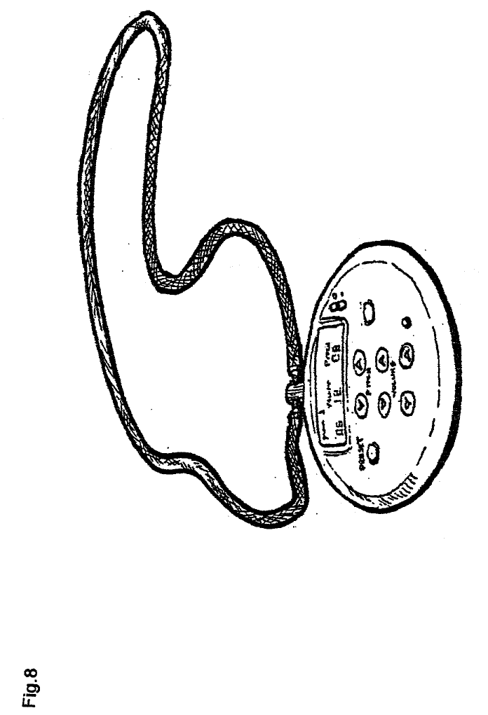

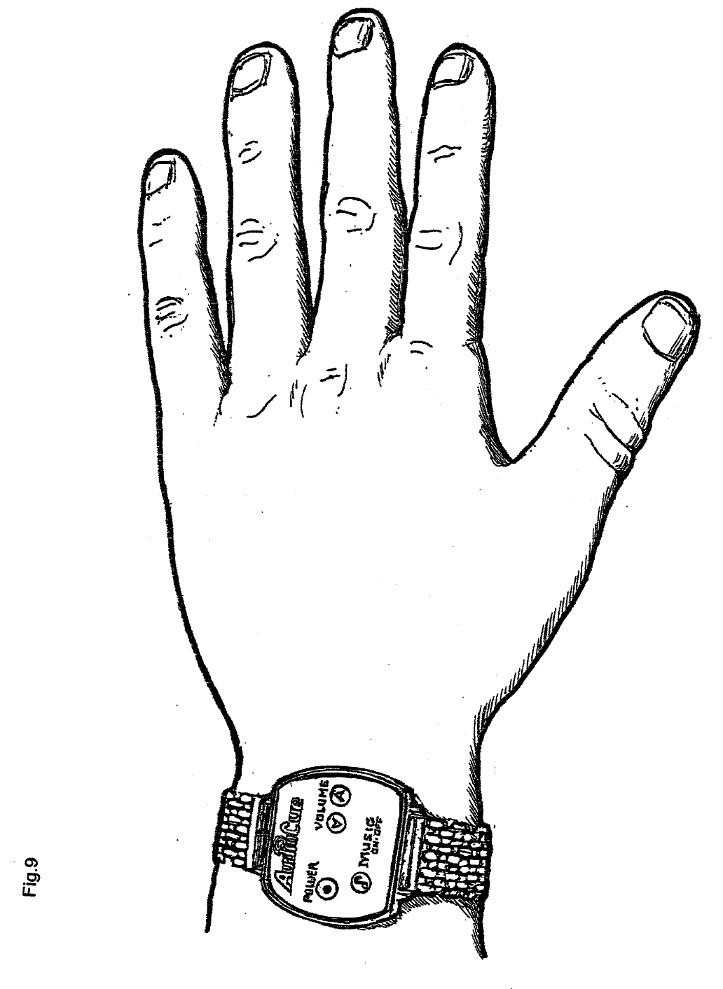

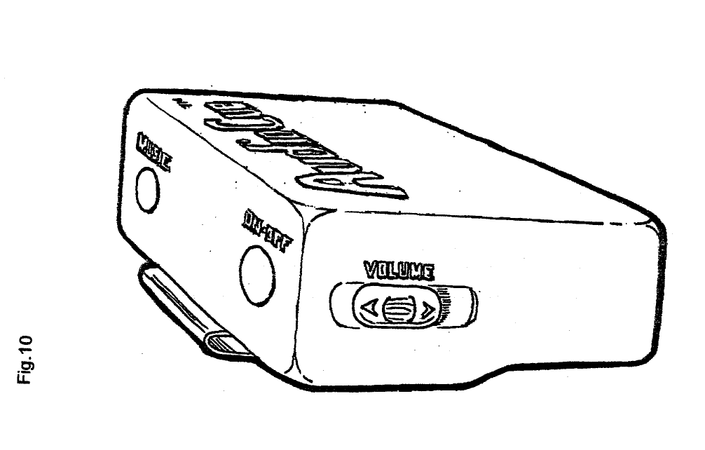

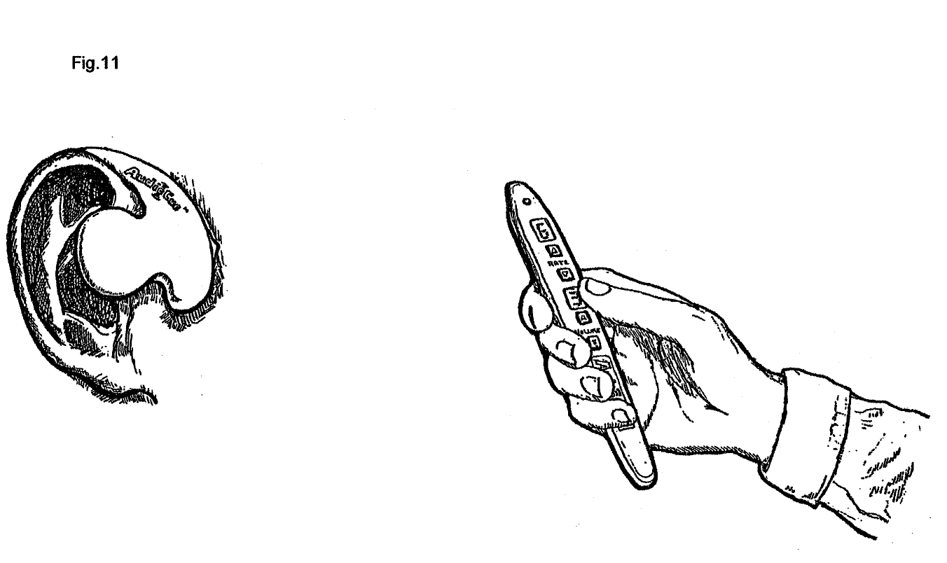

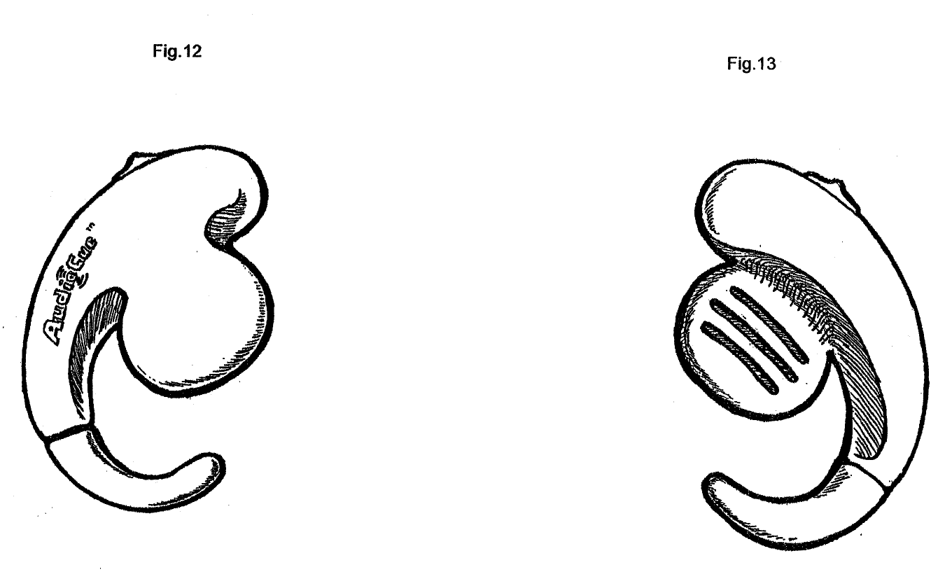

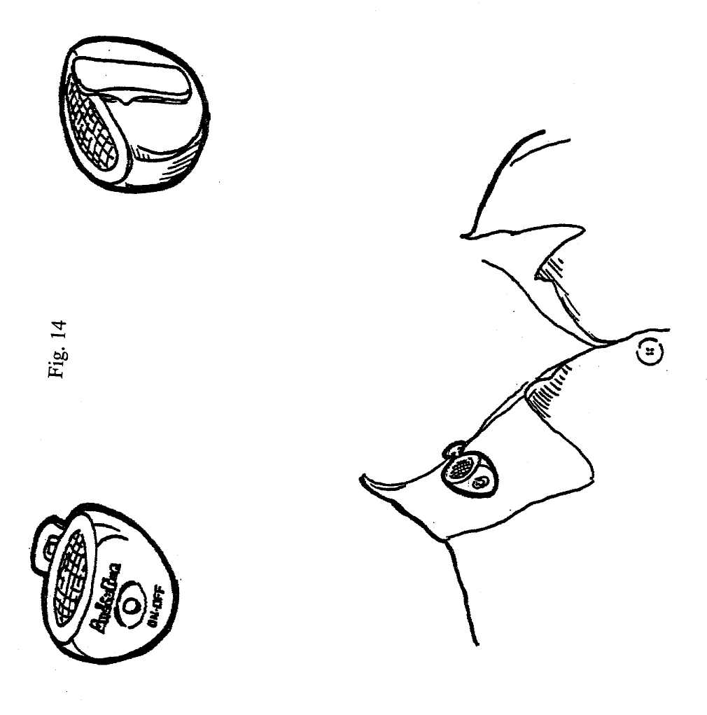

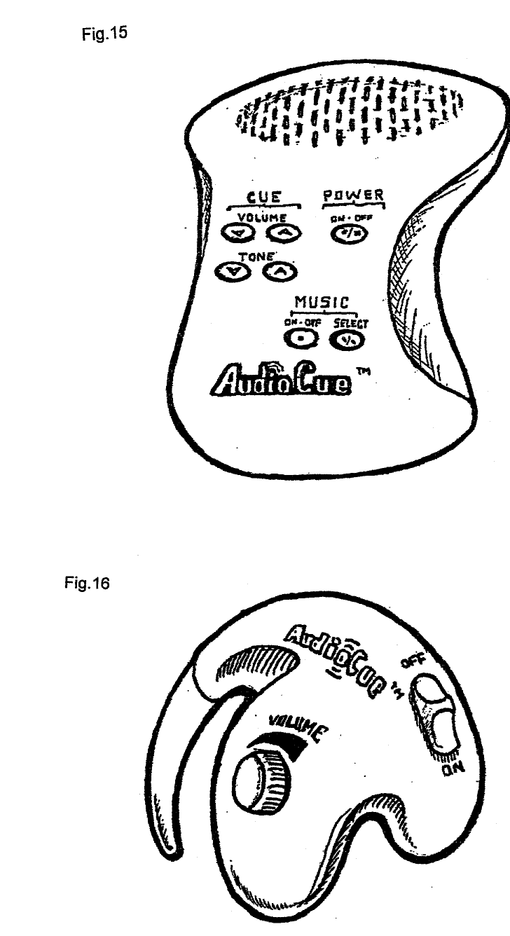

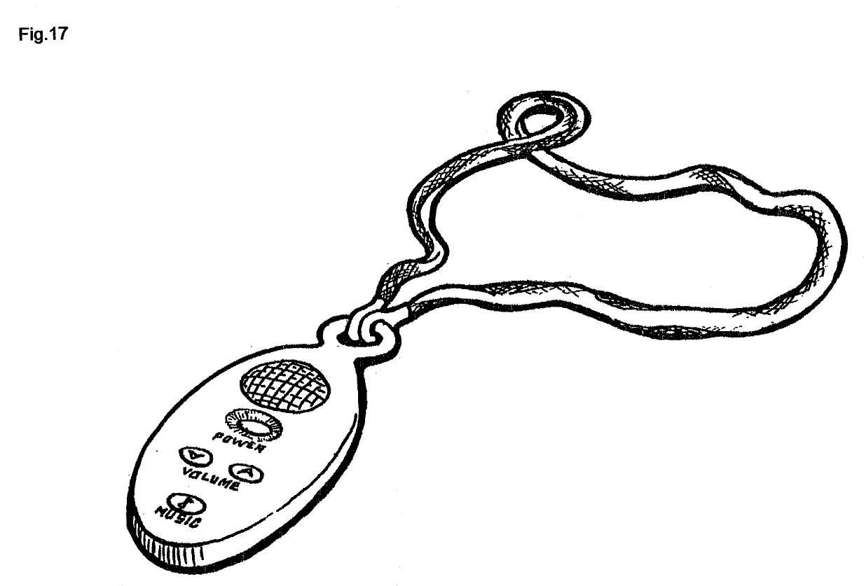

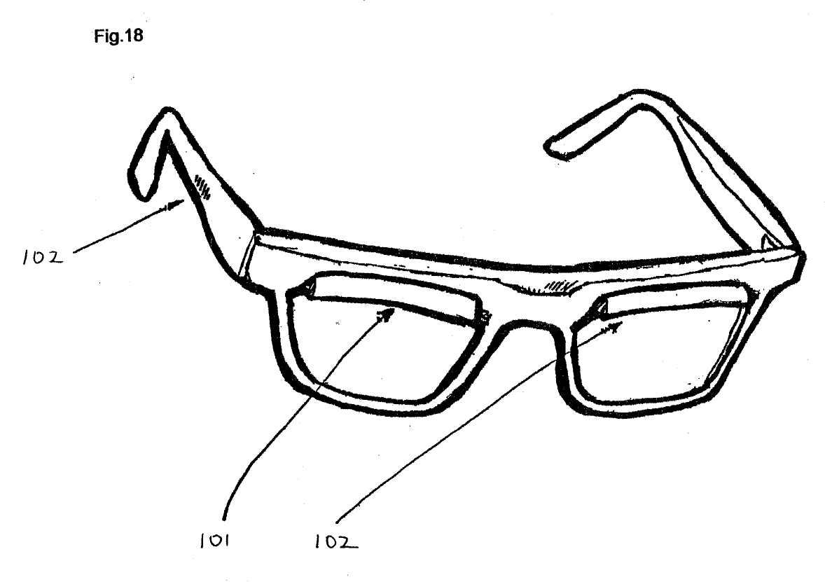

The architecture and subsystems of the present invention are shown in FIGS. 1-4; various embodiments of the device housing, configuration, appearance and physical design are shown in FIGS. 5-18. In its broadest, most general aspect, the present invention provides a device 10 for movement initiation, or rhythmic stimulus, comprising at least two main systems, namely, a control system 20 and an output system 30, that work together to generate a desired audio, visual, or other type of output. The preferred embodiment of the present invention generates rhythmic stimuli in the form of auditory outputs. The first system comprises an auditory output unit 40, as shown in FIG. 3, that transmits or projects the actual rhythmic auditory stimuli to the cochlea 41 for treatment of patients with diseases, symptoms, and or syndromes such as Parkinson's disease and Parkinsonism. The second system is a user controller 50, shown in FIG. 2, that sends commands, signals, and programs to the auditory output unit 40, as shown in FIG. 3, to control within certain limits the audio output of the rhythmic stimuli based on the parameters that have been programmed into the user controller 50. A clinician or master controller 60, shown in FIG. 1, that uniquely programs the user controller for use by a specific patient may be added as a third system. The added clinician controller 60 will allow the clinician to input into the user controller 50 the appropriate rhythmic stimuli treatment options based on what the clinician has found through trial and error to be most beneficial for the patient. Thus, the clinician will continue to maintain a certain degree of control over the patient's treatment when it is being administered through use of the user controller 50 outside of a clinical setting and not in the presence of a clinician. Alternatively, the clinician or master controller 60 may replace the user controller 50 as one of the main two systems, especially if the clinician prefers not to allow the patient to administer his or her treatments independently through the user controller 50. This set up allows the clinician to have complete control over the administered treatments for a specific patient. In this case, the clinician controller 60 would function identical to the user controller 50 but may have expanded programming capabilities with respect to the audio output unit 40. These systems may be separately housed from one another or may be sub-systems integrated together to make up one or more systems enclosed by a single or dual housing.

In a first embodiment of the present invention, the movement initiation device 10 of the present invention consists of the three main systems described above. The clinician controller 60, the user controller 50, and the audio output unit 40 are individual systems that each have their own role in providing the most beneficial and or desired rhythmic stimuli for the patient being treated.

The clinician controller 60, shown in FIG. 1, is preferably a hand holdable or tabletop unit that is to be used by the clinician treating the patient. The clinician controller 60 may also be a personal computer 62 or other programmable computing device that can be programmed appropriately and interfaced to externally. The clinician controller 60 stores a multitude of programs and parameters that can be programmed into the user controller 50 or downloaded to the audio output unit 40 upon the clinician's input. The clinician controller 60 allows the clinician to control the rhythmic stimuli that are programmed into the user controller 50 and thus, control the user's or patient's rhythmic stimuli treatment options during everyday life when the patient is outside the clinical setting. The internal design of the clinician controller 60 includes, but is not limited to, a central processing unit 64, non-volatile memory 66 and an output 68 that has a wired or wireless (remote) connection to the central processing unit (described hereinbelow) of the user controller 50 and or the audio output device 40. The personal computer 62 can comprise the central processor 4, memory 66, and input processor 72. Programs of rhythmic sounds, musical tunes and their parameters, such as volume, tempo, duration, etc, are downloaded via a wired or wireless input port into the memory 60, where they are stored until retrieved upon command from the user interface 70. The user interface 70 is accessible by the clinician along the exterior of the clinician controller 60 in the form of buttons, keypads, touch-pads or similar components, or through a computer interface such as a keyboard, touch-screen, mouse, or other typical human-computer interface device. The commands directed from the user interface 70 are sent to the central processing unit 64 by the input processor 72, which then extracts the chosen programs and parameters from the memory 66 and directs them through the output 68 to the input or central processing unit of the user controller 50 or directly to the audio output unit 40. If the clinician controller 60 is directly interfacing with the audio output unit 40, then it directs the commands to the audio output unit 40 without the assistance of the user controller 50. The clinician controller 60 may be preprogrammed and additional programming may be downloaded at any time through downloads from a computer interface or other sources of programming at the discretion of the clinician.

The user controller 50, as shown in the schematic diagram of FIG. 2, is a device used by the patient, caregiver, or physician and can be a tabletop unit, a hand-held unit, or a device that straps to, attaches to, hangs from, or is otherwise interfaced to the body. The user controller 50 directs commands and other information from its external user interface 70 and regulates the operation of the auditory output unit 40 through a wired or wireless (remote) interface between the two systems, as shown in FIG. 4. Such controls that may be operable from the user interface include on/off, volume, rhythm or tune, tempo, pitch, frequency and duration of both individual tones and the entire sound production. These controls may include buttons, keypads, touch pads, joysticks and or an external computer interface, or other similar components. To assist users with restricted mobility of the arms and hands, the controls may also be activated by voice or movements of the user's body including, but not limited to, head, eye, limb, finger/toe, hand/foot, or tongue movements and/or myoelectric signals produced by these movements.

The internal design of the user controller 50 comprises a central processing unit 76, non-volatile memory 78, a wired or wireless input 80 from the clinician controller 60 and an output 82 that has a wired or wireless (remote) connection to the central processing unit of the audio output unit (discussed hereinbelow). Programs of rhythmic sounds, musical tunes and their parameters, such as volume, tempo, frequency, duration, and the like, are downloaded from the clinician controller 60 into the user controller memory 80 where they are stored until retrieved upon command from the user interface 74. Alternatively, if the user controller 50 does not interface with a clinician controller 60, it may be preprogrammed and additional programming may be downloaded at any time through downloads from the computer interface or other sources of programming at the discretion of the clinician or patient. The commands directed from the user interface 74 are sent to the central processing unit 78, which then extracts the chosen programs and parameters from the memory 80 and directs it through the output 82 to the input or central processing unit of the audio output unit 40. The user controller 50 may be activated by the push of a button (power on/off button), or may be activated by sound, such as clapping or speech. An optional display 84 permits monitoring current program status and parameter values.

The audio output unit 40 comprises a central processing unit 86, non-volatile memory 88, a clock or oscillator 90, an audio synthesizer 92 and a signal output transducer 94. The central processing unit 86 interprets the inputs from the clinician controller 60 or the user controller 50 and is responsible for saving programs and other information to the memory 88 and executing commands with the assistance of the other system components. The tune memory 96 stores rhythmic sounds, musical tunes and their parameters, such as tempo, volume, frequency, duration, and the like received from the input processor 97, which in turn receives signals from the user controller 50, until they are recalled and extracted by the central processing unit 86. The speed or timing of the rhythm may be significant for specific patients for producing effective results. The clock or oscillator 90 produces the synchronizing timing signal for the system to produce the programmed tempo or duration of the individual rhythmic sounds and of the entire sound production. To produce the desired audio output as commanded by the clinician or user controller 60, 50, the appropriate sounds and parameters extracted from the memory 88, 96, are communicated by the central processing unit 86 to the audio synthesizer 92 for the production of the sound waves. These sound waves are converted into sound via an output transducer 94. The sound production is continuous until the system is turned off or for the programmed amount of time, such as 30 to 60 seconds, as controlled by the timing circuit, or internal clock 90, and then it automatically shuts down. An amplifier or speaker may be included to improve the detectability of the sound by the patient.

Additionally, the audio output unit synthesizer 92 and possibly portions of the processor 86 and memory 88, 96 may be replicated to produce a multi-part musical composition. In this case, the various outputs would be summed or combined to produce the desired output. Also, the volume could be changed dynamically or an additional signal could be superimposed on the output to produce desired signals, such as accentuating the downbeat of the music.

When the audio output unit 40 is separately housed from the clinician and user controllers 60, 50, it may be completely controlled by the clinician or user controller or alternatively, may have limited controls accessible by the patient for powering itself on and off and for adjusting the volume at which the audio is projected.

For portability and convenience, the audio output unit 40 may be carried on a person's body. For example, it may be wearable around the neck or wrist or may consist of small circuitry capable of fitting inside a ring, wrist or pocket watch, bracelet, pendent, collar clip-on, and the like.

The audio output unit 40 may be a modified hearing aid that, in addition to providing hearing capabilities, produces rhythmic auditory stimulation. This two-in-one device assists those Parkinson's patients with hearing impairments as well as generates rhythmic auditory patterns to treat symptoms of Parkinson's disease and Parkinsonism. The hearing aid portion of this audio output unit 40 may be of an existing, commercially available design (external, internal or partially internal hearing aid) or may be uniquely designed to coexist with the audio output system. To minimize the electronic circuitry and thus the housing size, the hearing aid and the audio output unit may be incorporated into one microchip. The audio output/hearing aid device may have separate control knobs or buttons for adjusting the amplification of the hearing aid and for adjusting the level of sound present to the patient's ear so that the audio output and hearing aid systems can each function separate of the other.

Whether or not the audio output unit 40 functions as a hearing aid, it may be packaged in an ear wearing device, see, for example, but not by limitation, FIGS., 12, 13 and 16, such as a hearing aid-like housing, to aid in the comfort level and convenience of wearing such a device and to maximize the transmission from the output to the auditory system of the patient. The ear wearing device may be worn externally, internally or partially internally and may take the form of any of the hearing aids or other ear wearing devices that are commercially available or may be unique to this invention. As another example, the audio output unit may be integrated into a set of wired or wireless headphones for ease of use and less obtrusive noise for others.

The possibility of multiple arrangements of the clinician controller 60, user controller 50 and audio output unit 40 can create additional embodiments of the present invention. For example, the clinician controller 60, user controller 50, and audio output unit 40 may all reside within one tabletop or hand holdable device as seen in the attached FIGS. 5-11. Alternatively, one of the clinician controller 60 or user controller 50 systems may be combined with the audio output unit 40 to create a single portable device that can be programmed by the clinician, accessed by the patient when outside the clinical setting, and can generate the rhythmic sound as programmed for patient treatment. An advantage of this embodiment is that one does not have to keep track of multiple devices and assure that they are all operable and have properly working wired or wireless connections. Through pass codes and control locks, the clinician can still control the patient's treatments to the extent the clinician feels is necessary. The clinician or user can be the programmer, or if the clinician prefers, the patient may have only limited control over the prescribed treatment (patient may only be allowed to operate the device or the patient may be allowed limited control over the treatment by controlling volume and duration). In another embodiment, the clinician and user controllers 60, 50 may be combined into one device and the audio output unit 40 separately housed. This allows the audio output unit 40 to be in the form of an ear-wearing device so that the projected sound is less obtrusive to others and has less interference from ambient noise. It may be redundant to have both the clinician and user control systems in a single housing, so they may be combined into one control system. Yet another example is the combination of the user controller and the audio output into a single housing with the clinician controller being separate or non-existent. A further embodiment consists of two main systems, the clinician controller and the audio output unit, being housed together. One example is that the clinician controller, user controller and audio output unit are each their own separate device that work together to create a three piece apparatus; e.g., earpiece containing the output device 40, a belt clip or other wearable unit containing the user controller 50, and, a clinician controller 60.

As described previously, the rhythmic stimuli generated and controlled by the present invention is not limited to auditory cues. Visual, tactile or other type cues can also be programmed into the system. Visual cues may be in the form of rhythmic patterns of visual light or illumination, which may be created by cascading beams of light, projected laser beams, flashing lights, LED's, and the like, projected from a projection device 101 associated with the frame, as shown in FIG. 18. For portability and convenience, a pair of prescription or sunglasses can be the source of visual output. Light beams are projected in a rotating pattern onto the lenses, such that they simulate a moving sidewalk for initiating the physical response of walking. Tactile cues may be in the form of rhythmic tapping on the body. Vibratory cues may be in the form of physical/active vibrations, which are conducted through bones or other structures or tissues in the human body. The movement initiation device may be programmed with more than one type of rhythmic stimuli to further enhance treatment. The output device would then have to be such that it would be capable of generating all programmed rhythmic stimuli. For example, to generate both visual and aural stimulation, the stimuli output device may be a pair of the above-identified glasses, shown in FIG. 18, with a speaker 102 embedded in the temporal arms or with an audio output unit attached thereto.

Additional applications for the present invention include its use by musicians for tuning their instruments and singing voices. It can be used as a portable metronome or tuner for enhancing one's singing or musical instrument ability. As a tuning device, it matches the tone of the instrument to the audio output and as a portable metronome, it times the beats and keeps the musician from straying from the desired rhythm. This tuning and rhythmic counting device could be used by an individual or a group, such as an orchestra. Each member of the orchestra has an audio output unit, preferably housed within a small earpiece that can be turned on simultaneously and with the same output as the others via remote control.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

Any of the above described embodiments of the present invention may be used in other applications where rhythmic stimuli can be beneficial, especially for improving motor control which is negatively affected by certain medical problems.

Claimed is:

1. A device for producing rhythmic stimuli to a user for assisting in the continuing movement of the user's muscles, comprising:
a. a housing;
b. a first controller for use by said user, said first controller comprising
i) nonvolatile memory, and
ii) a central processing unit;
c. a second controller for programming said first controller; said second controller comprising
i) nonvolatile memory,
ii) a central processing unit,
iii) means for loading data into said second controller nonvolatile memory,
iv) an output device for communicating with said data processing device, and
v) a user interface;
d. an output device comprising
i) nonvolatile memory,
ii) means for producing a synchronizing timing signal,
iii) an audio synthesizer, and
e. means for creating a detectable signal;
f. a central data processing device comprising
i) nonvolatile memory, and
ii) a central processing unit
iii) whereby said central data processing device interprets input signals from said first and said second controllers and executes commands.

2. The device of claim 1, wherein said housing is adapted to be worn in proximity to an ear of said user.

3. The device of claim 1, wherein said nonvolatile memory of said central data processing device comprises a memory area for storing rhythmic sounds, musical tunes and accompanying parameters.

4. The device of claim 3, wherein said parameters are selected from the group consisting of tempo, volume, frequency, and duration of said rhythmic sounds and mixtures thereof.

5. The device of claim 3, wherein said nonvolatile memory of said central data processing device comprises a memory area for storing saving programs and other information.

6. The device of claim 1, wherein said means for creating a detectable signal is a transducer.

7. A device for producing rhythmic stimuli to a user for assisting in the continuing movement of the user's muscles, comprising:
a. a housing adapted to be worn in proximity to an ear of said user;
b. a first controller for use by said user, said first controller comprising
i) nonvolatile memory, and
ii) a central processing unit;
c. a second controller for programming said first controller; said second controller comprising
i) nonvolatile memory,
ii) a central processing unit,
iii) means for loading data into said second controller nonvolatile memory,
iv) an output device for communicating with said data processing device, and
v) a user interface;
d. an output device comprising
i) nonvolatile memory,
ii) means for producing a synchronizing timing signal,
iii) an audio synthesizer,
e. a transducer; and,
f. a central data processing device comprising
i) nonvolatile memory comprising
(1) a memory area for storing rhythmic sounds, musical tunes and accompanying parameters, wherein said parameters are selected from the group consisting of tempo, volume, frequency, and duration of said rhythmic sounds and mixtures thereof, and
(2) a memory area for storing saving programs and other information,
ii) a central processing unit
iii) whereby said central data processing device interprets input signals from said first and said second controllers and executes commands.
